(19) United States
(12) Patent Application Publication
(10) Pub. No.: US 2008/0221649 A1
(43) Pub. Date: Sep. 11, 2008

(54) Method of sequentially treating tissue

(76) Inventors: Agustina Echague, Milton, MA (US); Jayant D. Bhawalkar, Brighton, MA (US); James C. Hsia, Weston, MA (US)

Correspondence Address: PROSKAUER ROSE LLP, ONE INTERNATIONAL PLACE, BOSTON, MA, 02110, US

(21) Appl. No.: 11/716,395

(22) Filed: Mar. 9, 2007

(51) Int. Cl. A61N 5/00

(52) U.S. Cl. 607/100

(57) ABSTRACT

A treatment for deep tissue using long effective pulse durations is described. Fatty tissue can be treated by delivering a beam of radiation to a subcutaneous fat region disposed relative to a dermal interface in a target region of skin. Radiation is delivered to a first region of tissue as by exposure to pulses of the beam of radiation in a stacked fashion. Between successive exposures of the first region of tissue, other regions of tissue are exposed to the beam of radiation.

### FIELD OF THE INVENTION

The invention relates generally to cosmetic treatments, and more particularly to using a beam of radiation to target deep tissue with an effectively long pulse duration.

### BACKGROUND OF THE INVENTION

Many light-based and RF-based treatments of skin disorders require long exposure times. Examples of such treatments are those that target deep tissue while maintaining overlying skin at a relatively low temperature. These treatments can be used for several purposes such as treating cellulite, treated vascular problems, such as ablating varicose veins, encouraging skin pigmentation, and skin rejuvenation.

In some dermatological treatments using light or RF resources, a long exposure time in the range of many seconds is desirable. Longer exposure of a deeply penetrating wavelength and skin surface cooling can lead to deeper thermal heat zone within the skin. Hence, deeper structures can benefit from longer exposure. Furthermore, if radiation is applied to the surface of the skin over a long time period, the skin can remain cooler compared to a short exposure because fatty tissue generally has poorer heat conductivity and it is relatively poorly perfused with blood flow. However, such long exposure durations can make treatments very slow and impractical.

One way to increase treatment speed is to make the treatment spot larger. However, there is a practical limit to the spot size depending on the contours of the area of the body to be treated. Also, a larger spot may be more painful because more nerves are stimulated.

### SUMMARY OF THE INVENTION

The invention, in one embodiment, features a treatment for deep regions of the skin having a slower thermal relaxation time than an overlying tissue. This may include a fatty deposit located in, proximate to, above or below a dermal interface. Instead of being an invasive surgical procedure, treatment radiation is directed through the surface of the skin. A treatment can make use of the thermal properties of the deep tissue. For instance, where the target tissue is fat, the thermal conductivity of the fat is lower than the average dermis, causing it to cool slower than the average dermis or epidermis after heating. Subcutaneous fatty tissue has a longer thermal relaxation time, which can be exploited by allowing targeted fatty tissue to retain a higher temperature between successive exposures to treatment radiation without requiring continuous exposure to the treatment radiation during the treatment.

The process of applying a sequence of time-spaced pulses having a relatively small fluence can result in an effectively long pulse duration with a larger total fluence. The process also allows the dermis to remain cooler during treatment, resulting in lower pain while enabling the target fat temperature to rise to therapeutic temperatures. The time between exposing an area of tissue to successive pulses can be used to expose other regions of the treated tissue to pulses of the radiation.

Cooling can be used to protect the skin surface, to minimize unwanted injury to the surface of the skin, and to minimize any pain that a patient may feel. An additional advantage of such a treatment is that the treatment can be performed with minimal acute cosmetic disturbance such that the patient can return to normal activity immediately after the treatment.

In one aspect, the invention features a method of applying energy to a plurality of regions of a biological tissue by exposing a first region of the biological tissue to an electromagnetic beam for a first selected length of time, positioning the electromagnetic beam over a second region of the biological tissue, and exposing the second region of the biological tissue to the electromagnetic beam for a second selected length of time. Subsequently, the method positions the electromagnetic beam over the first region of the biological tissue and exposes the first region of the biological tissue to the electromagnetic beam for a third selected length of time. In various embodiments, the method is repeated for a preset time or for a preset number of times or until a therapeutic temperature is reached. In various embodiments, the method damages at least one fat cell and/or cause partial denaturation of collagen fibers in a dermal zone. In various embodiments, the method is repeated by exposing a multiple regions of the biological tissue using predetermined temporal sequence.

In another aspect, the invention features a method of applying energy to a plurality of regions of a biological tissue by exposing a first region of the biological tissue to an electromagnetic beam for a first length of time. The first region includes a first tissue zone overlying a second tissue zone. The method further positions the electromagnetic beam over a second region of the biological tissue and exposes the second region of the biological tissue to the electromagnetic beam for a second length of time, during which the first tissue zone cools substantially more than the second tissue zone. The method further repositions the electromagnetic beam over the first region of the biological tissue and re-exposes the first region of the biological tissue to the electromagnetic beam before the second tissue zone cools below a threshold temperature. In various embodiments, the method is repeated for a preset time or for a preset number of times or until a therapeutic temperature is reached. In various embodiments, the method damages at least one fat cell and/or cause partial denaturation of collagen fibers in a dermal zone. In various embodiments, the method is repeated by exposing a multiple regions of the biological tissue using predetermined temporal sequence.

In yet another aspect, the invention features a method for applying energy to a first biological tissue that has a longer thermal relaxation time than a second biological tissue which is above the first biological tissue. The method exposes a first region of the first and the second biological tissues to an electromagnetic beam for a first length of time. The first length of time is short enough to prevent a temperature of the first region of the second biological tissue from substantially exceeding a first threshold value. The method further exposes a second region of the biological tissues to the electromagnetic beam for the first length of time, while the temperature of the first region of the second biological tissue decreases. The method further exposes the first region of the biological tissues to the electromagnetic beam for the first length of time, while a temperature of the second region of the second biological tissue decreases. The method repeatedly performs the exposing steps such that a temperature of the first region of the first biological tissue and a temperature of the second region of the first biological tissue exceeds a second threshold value, whereas the temperature of the first region of the second biological tissue and the temperature of the second region of the second biological tissue do not substantially exceed the first threshold value. In various embodiments, the method is repeated for a preset time or for a preset number of times or until a therapeutic temperature is reached. In various embodiments, the method damages at least one fat cell and/or cause partial denaturation of collagen fibers in a dermal zone. In various embodiments, the method is repeated by exposing a multiple regions of the biological tissue using predetermined temporal sequence.

In various embodiments, the beam of radiation can be delivered to the target region up to about 10 mm below the surface of the skin. In some embodiments, the beam of radiation can be delivered to the target region about 0.5 mm to about 10 mm below the surface of the skin. The target region of the skin can be between about 1 mm and about 5 mm below the surface of the skin. The target region of the skin can be between about 0.5 mm and about 2 mm below the surface of the skin.

Other aspects and advantages of the invention will become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows a three dimensional cutaway view of skin including subcutaneous tissue being treated by a beam of radiation.

FIG. 2A depicts an exposure pattern for repeatedly exposing a treatment area to multiple radiation pulses.

FIG. 2B depicts another exposure pattern for repeatedly exposing a treatment area to multiple radiation pulses.

FIG. 2C depicts yet another exposure pattern for repeatedly exposing a treatment area to multiple radiation pulses.

FIG. 3 shows an exemplary system for treating deep tissue.

FIG. 4 depicts a planoconvex lens positioned on a skin surface.

FIG. 5 shows a plurality of lens focusing radiation to a target region of skin.

FIG. 6 shows a lens having a concave surface positioned on a skin surface.

FIG. 7A shows a plan view of a laser diode array.

FIG. 7B shows an enlarged perspective view of the laser diode array of FIG. 7A.

FIG. 8 shows a handpiece of an ultrasound device placed proximate to a skin surface.

FIG. 9 shows a hand piece for treating fatty tissue using multiple beams of radiation.

FIG. 10 shows the intensity as a function of time of a radiation source and the exposure intensity as a function of time of some regions of tissue.

### DESCRIPTION OF THE INVENTION

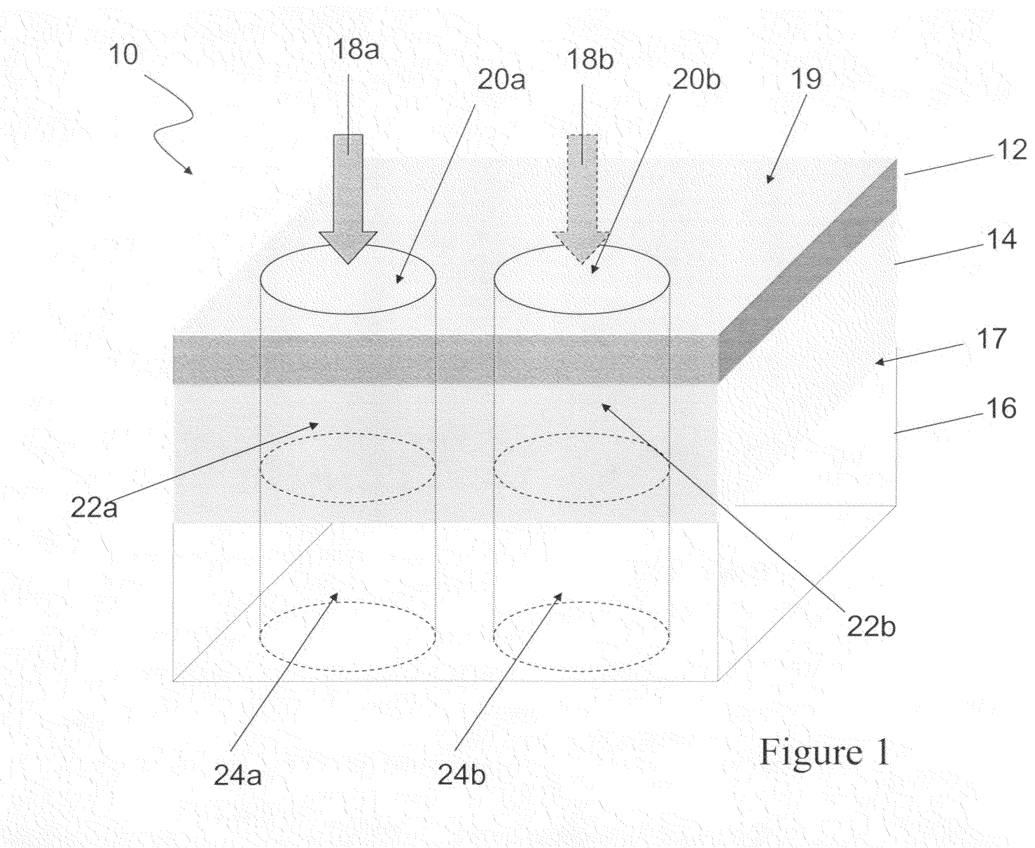

FIG. 1 shows a cross-section of skin 10 including an epidermal layer 12, a dermal layer 14, a region of deep tissue 16 to be treated, and a dermal interface 17. In some embodiments, the deep tissue 16 includes fatty tissue or subcutaneous fat. The deep tissue 16 need not be a separate layer of the skin, and can instead be a lower portion of the dermal layer 14. The deep tissue 16 can, for example, include veins, fat, cellulite, or the like, which can be targeted.

The deep tissue 16 can be treated by applying several pulses of radiation having short pulse durations to several zones within the deep tissue 16 treatment area. By directing subsequent pulses of radiation to the several zones, each zone of tissue can be exposed to an effective long pulse duration. having a large cumulative fluence. Using this technique, larger total fluence can be delivered to the deep tissue 16 than would otherwise be available if radiation was delivered continuously to the deep tissue 16. Tissue overlying the deep tissue 16 can be allowed to partially or fully thermally relax between successive pulses, during which time the beam of radiation can be used to expose other zones of the deep tissue 16.

Subcutaneous fat and/or cellulite can be treated by injuring fatty tissue (e.g., a fatty deposit located at or proximate to the dermal interface 17). In various embodiments, a treatment can, for example, reduce fat, remove a portion of fat, improve skin laxity, tighten skin, strengthen skin, thicken skin, induce new collagen formation, promote fibrosis of the dermal layer or subcutaneous fat layer, or be used for a combination of the aforementioned. Cellulite can result from fatty tissue which can permeate or cross the dermal interface 17 and invade the dermal layer 14.

A beam of radiation 18 can be used to treat at least a portion of the deep tissue 16 by delivery through a surface 19 of the epidermal layer 12. The radiation beam 18 can be applied to several regions within a target region, for example, a first beam 18a can target a first region 20a and a second beam 18b can target a second region 20b. In some embodiments, radiation 18x can be applied in a collimated fashion, resulting in a cylindrical exposure region. In some embodiments, radiation 24 can be applied in a non-collimated fashion, resulting in a non-cylindrical exposure region.

Each region 20x of tissue that is exposed can be further divided into multiple tissue zones. In some embodiments, the first region 20a includes a first tissue zone 22a and a second tissue zone 24a. The second region 20a includes a first tissue zone 22b and a second tissue zone 24b. The second tissue zone 24x can underlie the first tissue zone 22x. In some embodiments, the second tissue zone 24x can be at and/or below the dermal interface 17.

In various embodiments, radiation beam 18a exposes tissue region 20a, thereby delivering thermal energy to tissue zones 22a and 24a, during a radiation pulse. The radiation beam can be moved so that radiation beam 18b exposes a tissue region 20b, thereby delivering thermal energy to tissue zones 22b and 24b, during a radiation pulse. Tissue region 20a can be allowed to partially or fully thermally relax while tissue region 20b is being exposed. This step can be repeated for other tissue regions (not shown) while tissue regions 20a and 20b partially or fully thermally relax. The radiation beam can be moved so that radiation beam 18a re-exposes tissue region 20a, thereby delivering thermal energy to tissue zones 22a and 24a, during a radiation pulse. This radiation beam movement can be repeated so that each tissue zone 22a and 24a receives multiple pulses of radiation. The radiation pulses can be relatively short as compared to the total exposure time, so that an effective long pulse duration results.

The process of repeatedly exposing various tissue regions 20x can be repeated until a desired effect, such as coagulation, vaporization, partial denaturation, denaturation, or ablation of tissue, in the tissue region occurs. In certain embodiments, the effect occurs in the tissue zones 24x of the tissue 16. The overlying tissue zones 22x need not undergo a tissue effect, such as coagulation, vaporization, partial denaturation, denaturation, or ablation, can undergo a low level thermal effect, or can undergo coagulation, vaporization, partial denaturation, denaturation, or ablation to a degree less severe than the degree of treatment to the tissue zones 24x. For example, tissue zones 22x can partially or fully thermally relax while tissue zones 24x is being treated.

The treatment radiation can damage one or more cells of the deep tissue 16. In some embodiments, damage to the deep tissue 16 causes at least a portion of lipid contained within a fat cell to escape or be drained from the treatment area. At least a portion of the lipid can be carried away from the tissue through biological processes. In one embodiment, the body's lymphatic system can drain the treated fatty tissue from the treated region. In an embodiment where a fat cell is damaged, the fat cell can be viable after treatment. In one embodiment, the treatment radiation can destroy one or more fat cells. In one embodiment, a first portion of the fat cells is damaged and a second portion is destroyed. In one embodiment, a portion of the fat cells can be removed to selectively change the shape of the body region.

In some embodiments, the beam of radiation can be delivered to the target tissue zone to thermally injure, damage, and/or destroy one or more fat cells. For example, the beam of radiation can be delivered to a target chromophore in the target region. Suitable target chromophores include, but are not limited to, a fat cell, lipid contained within a fat cell, fatty tissue, a wall of a fat cell, water in a fat cell, and water in tissue surrounding a fat cell. The energy absorbed by the chromophore can be transferred to the fat cell to damage or destroy the fat cell. For example, thermal energy absorbed by dermal tissue can be transferred to the fatty tissue. In one embodiment, the beam of radiation is delivered to water within or in the vicinity of a fat cell in the target region to thermally injure the fat cell.

In various embodiments, treatment radiation can affect one or more fat cells and can cause sufficient thermal injury in the dermal layer of the skin to elicit a healing response to cause the skin to remodel itself. This can result in more youthful looking skin and an improvement in the appearance of cellulite or other cosmetic defects. In one embodiment, sufficient thermal injury induces fibrosis of the dermal layer, fibrosis on a subcutaneous fat region, or fibrosis in or proximate to the dermal interface. In one embodiment, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target tissue zone, as well as thicken the skin, tighten the skin, improve skin laxity, and/or reduce discoloration of the skin.

In various embodiments, a zone of thermal injury can be formed at or proximate to the dermal interface. Fatty tissue has a specific heat that is lower than that of surrounding tissue. For example, typical fatty tissue has a volumetric specific heat of about 1.8 J/cm³ K, whereas typical skin has a volumetric specific heat of about 4.3 J/cm³ K. As the target region of skin is irradiated, the temperature of the fatty tissue exceeds the temperature of overlying and/or surrounding dermal or epidermal tissue. The temperature within the fatty tissue can generally rise faster than the temperature within the overlying dermal tissue.

In various embodiments, a cumulative fluence of radiation is desired for treatment. This cumulative fluence can be achieved by successive exposure of each tissue zone to pulses of a beam of radiation having a fluence less than the desired cumulative fluence of the treatment. When targeting deep zones of the skin such as subcutaneous fat, various embodiments may significantly speed up treatment. Fat generally has a lower thermal conductivity than the average dermis. Fat may therefore cool much slower than the average dermis or epidermis after heating. Fat has a longer thermal relation time than the overlying dermis. This property can be used effectively to enhance selective heating of fat compared with the overlying dermis and epidermis. This effect is further enhanced by the faster heat removal from the dermis by blood flow and/or by cooling. Longer treatment durations can benefit from this effect because the fat may retain its heat longer and the dermis may cool faster, effectively increasing the temperature differential between the fat and the dermis, which may reduce pain or discomfort. Biological tissues having a longer thermal relaxation time than an overlying biological tissue can be treated. Fatty tissues having a longer thermal relaxation time than an overlying dermal tissue can be treated.

Effective long pulse durations can be applied as a sequence of time-spaced short pulses rather than a continuous exposure to the radiation beam. A temperature gradient between the dermis and the fat gets progressively larger with each pulse. The temperature gradient between the fat and the dermis is increased during each pulse, for instance, because the fat has a lower volumetric specific heat than the overlying the dermis, causing the fat to heat more quickly. The temperature gradient between the fat and the dermis is also increased between each successive pulse, for instance, because the fat has a longer thermal relaxation time than the overlying the dermis, causing the fat to cool more slowly.

The process of applying a sequence of time-spaced pulses can be referred to as pulse stacking. When the energy contained in each pulse is small compared with the total energy delivered, the thermal effects can be equivalent to those of one continuous exposure. However, pulse stacking causes the dermis to remain cooler, resulting in lower pain while enabling the target fat temperature to rise to therapeutic temperatures.

While pulse stacking can produce similar tissue effects of a long treatment duration, pulse stacking may not substantially speed up the treatment if the radiation delivery device is held over the same treatment region until pulse stacking is complete. In various embodiments, the beam of radiation is moved from a first region of biological tissue to a second region of biological tissue between pulses and is later returned to the first region of biological tissue for subsequent exposure to a later pulse. The beam of radiation can be returned after the overlying tissue has dissipated heat. Stationary pulse stacking typically results in a relatively long pause between time-adjacent pulses of the radiation beam while the dermis cools. However, by moving the beam between time-adjacent pulses of the radiation beam, overlying tissue can cool while other regions of biological tissue are exposed to the radiation beam. The first region of biological tissue experiences pulse stacking when the beam of radiation is returned to the first region of biological tissue for subsequent exposure to a later pulse.

In various embodiments, the beam of radiation is moved between several regions of biological tissue in a predetermined pattern. As the predetermined pattern is repeated, the each region is thereby exposed to repeated pulses. Various embodiments use a thermal relaxation time between successive pulses of a stacked sequence at a first region of biological tissue to treat adjacent region of biological tissue. The treatment beam is applied to the treatment region of biological tissue long enough to apply a single pulse (within the desired stacked sequence). The beam can be moved in a substantially immediate fashion to a different region of biological tissue and a pulse is again applied. This process is repeated for multiple regions of biological tissue that cover a predetermined pattern on the skin, and the entire sequence is repeated. In this way, pulse stacking at each location is achieved but a larger area is treated in the same time than with stationary pulse stacking. Using this method can also result in reduced pain compared with one long exposure over a large area. Each region in the pattern is exposed to cumulative fluence greater than the fluence of the individual pulses.

In certain embodiments, the beam of radiation is moved manually, for instance by moving a hand piece manually. In certain embodiments, the beam of radiation is moved automatically, for instance by moving a hand piece electromechanically. For example, the sequential application of radiation can be done manually if the scan speed is slow, or using a mechanical scanner if the desired speed is high. The number of regions of biological tissue that are exposed before returning the beam of radiation to the first region of biological tissue, the number of times the regions of biological tissue are exposed during treatment, and the pulse length for each region can be adapted to the treatment, by considering such factors as source pulse duration, source pulse repetition rate, fluence of each pulse, total desired fluence, spot size of the beam of radiation, size of the treatment area, desired temperature of the targeted tissue, desired temperature of the dermis or other factors that would be apparent to one skilled in the art. An electromechanical scanner can include a pulse forming network and an optical scanner to assure that particular treatment zones are being revisited.

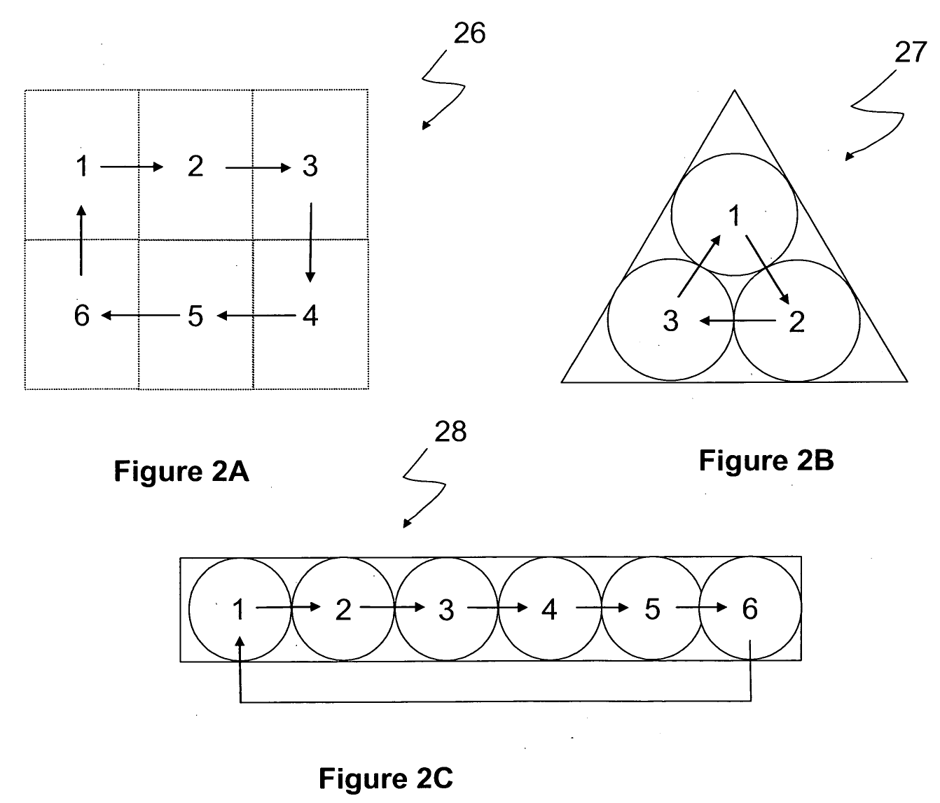

FIG. 2A shows one contemplated exposure pattern of successively exposing regions of the target tissue from a perspective looking into the plane of the skin to be treated. Exposure pattern 26 identifies a chronological order of regions of biological tissue to be exposed to pulses of the radiation beam. Exposure pattern 26 allows a rectangular portion of the treatment area to be exposed during treatment. Exposure pattern 26 can be repeated multiple times until the desired therapeutic effect is achieved.

FIG. 2B shows another contemplated exposure pattern of successively exposing regions of the target tissue in various embodiments from a perspective looking into the plane of the skin to be treated. Exposure pattern 27 identifies a chronological order of regions of biological tissue to be exposed to pulses of the radiation beam. Exposure pattern 27 allows a triangular portion of the treatment area to be exposed during treatment. Exposure pattern 27 can be repeated a number of times until the desired therapeutic effects are achieved.

FIG. 2C shows another contemplated exposure pattern of successively exposing regions of the target tissue in various embodiments from a perspective looking into the plane of the skin to be treated. Exposure pattern 28 identifies a chronological order of regions of biological tissue to be exposed to pulses of the radiation beam. Exposure pattern 28 allows a linear portion of the treatment area to be exposed during treatment. Exposure pattern 28 can be repeated a number of times until the desired therapeutic effects are achieved.

It will be appreciated that there are many other contemplated exposure patterns not shown in FIGS. 2A-2C. Furthermore, FIGS. 2C-2C show a limited number or regions in the pattern for illustrative purposes. It will be appreciated that the exposure patterns for use with various embodiments may also have more or less regions than are depicted.

In certain embodiments, the total treatment area is larger than the treatment area covered by a single instance of an exposure pattern, such as 26-28. In one embodiment, non-intersecting instances of an exposure pattern are repeated until the total treatment area has been treated. In one embodiment, intersecting instances of exposure pattern are repeated until the total treatment area has been treated. One effect of using intersecting instances of exposure pattern is that a single region of treated tissue, such as tissue region 20x, is exposed to pulses of radiation during subsequent intersecting instances of exposure pattern. In this way, an exposure pattern may be repeated while shifting the pattern until the total treatment area is treated. Subsequent applications of intersecting exposure patterns or repeated instances of a single exposure pattern result in pulse stacking for each region of biological tissue in the pattern.

In one embodiment, the peak temperature of the tissue can be caused to form at or proximate to the dermal interface. For example, a predetermined wavelength, fluence, pulse duration, and cooling parameters can be selected to position the peak of the zone of thermal injury at or proximate to the dermal interface. This can result in collagen being formed at the bottom of the dermis and/or fibrosis at or proximate to the dermal interface. As a result, the dermal interface can be strengthened against fat herniation. For example, strengthening the dermis can result in long-term improvement of the appearance of the skin since new fat being formed or untreated fat proximate to the dermal interface can be prevented and/or precluded from crossing the dermal interface into the dermis.

In one embodiment, fatty tissue is heated by absorption of radiation, and heat can be conducted into dermal tissue proximate the fatty tissue. The fatty tissue can be disposed in the dermal tissue and/or can be disposed proximate to the dermal interface. A portion of the dermal tissue (e.g., collagen) can be partially denatured or can suffer another form of thermal injury, and the dermal tissue can be thickened and/or be strengthened as a result of the resulting healing process. In such an embodiment, a fat-selective wavelength of radiation can be used.

In one embodiment, water in the dermal tissue is heated by absorption of radiation. The dermal tissue can have disposed therein fatty tissue and/or can be overlying fatty tissue. A portion of the dermal tissue (e.g., collagen) can be partially denatured or can suffer another form of thermal injury, and the dermal tissue can be thickened and/or be strengthened as a result of the resulting healing process. A portion of the heat can be transferred to the fatty tissue, which can be affected. In one embodiment, water in the fatty tissue absorbs radiation directly and the tissue is affected by heat. In such embodiments, a water selective wavelength of radiation can be used.

In various embodiments, a treatment can cause minimal cosmetic disturbance so that a patient can return to normal activity following a treatment. For example, a treatment can be performed without causing discernable side effects such as bruising, open wounds, burning, scarring, or swelling. Furthermore, because side effects are minimal, a patient can return to normal activity immediately after a treatment or within a matter of hours, if so desired.

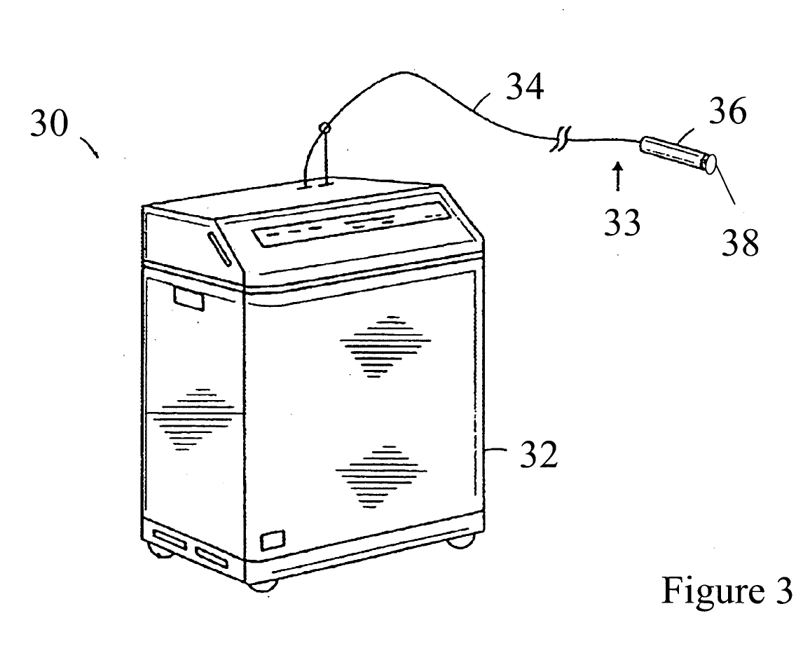

FIG. 3 shows an exemplary embodiment of a system 30 for treating tissue. The system 30 can be used to non-invasively deliver a beam of radiation to a target region. For example, the beam of radiation can be delivered through an external surface of skin over the target region. The system 30 includes an energy source 32 and a delivery system 33. In one embodiment, a beam of radiation provided by the energy source 32 is directed via the delivery system 33 to a target region. In the illustrated embodiment, the delivery system 33 includes a fiber 34 having a circular cross-section and a handpiece 36. A beam of radiation can be delivered by the fiber 34 to the handpiece 36, which can include an optical system (e.g., an optic or system of optics) to direct the beam of radiation to the target region. A user can hold or manipulate the handpiece 36 to irradiate the target region. The delivery system 13 can be positioned in contact with a skin surface, can be positioned adjacent a skin surface, can be positioned proximate a skin surface, can be positioned spaced from a skin surface, or a combination of the aforementioned. In the embodiment shown, the delivery system 33 includes a spacer 38 to space the delivery system 33 from the skin surface. In one embodiment, the spacer 38 can be a distance gauge, which can aid a practitioner with placement of the delivery system 33.

In various embodiments, the energy source 32 can be an incoherent light source, a coherent light source (e.g., a laser), a microwave generator, or a radio-frequency generator. In one embodiment, the source generates ultrasonic energy that is used to treat the tissue. In some embodiments, two or more sources can be used together to effect a treatment. For example, an incoherent source can be used to provide a first beam of radiation while a coherent source provides a second beam of radiation. The first and second beams of radiation can share a common wavelength or can have different wavelengths. In an embodiment using an incoherent light source or a coherent light source, the beam of radiation can be a pulsed beam, a scanned beam, or a gated continuous wave (CW) beam.

In various embodiments, the beam of radiation can have a wavelength between about 1000 nm and about 2,600 nm, although longer and shorter wavelengths can be used depending on the application. In some embodiments, the wavelength can be between about 1,000 nm and about 2,200 nm. In other embodiments, the wavelength can be between about 1,160 nm and about 1,800 nm. In yet other embodiments, the wavelength can be between about 1,190 nm and about 1,230 nm or between about 1,700 nm and about 1,760 nm. In one embodiment, the wavelength is about 1,210 nm or about 1,720 nm. In one detailed embodiment, the wavelength is about 1,208 nm, 1,270 nm, 1,310 nm, 1,450 nm, 1,550 nm, 1,720 nm, 1,930 nm, or 2,100 nm. One or more of the wavelengths used can be within a range of wavelengths that can be transmitted to fatty tissue and absorbed by the fatty tissue in the target region of skin.

In various embodiments, the treatment can deliver a beam of radiation with a cumulative fluence between about 1 J/cm² and about 500 J/cm², although higher and lower fluences can be used depending on the application. In some embodiments, the cumulative fluence can be between about 10 J/c^{m2} and about 150 J/cm². In one embodiment, the total cumulative fluence is between about 35 J/cm² and about 100 J/cm². In various embodiments, treatment of comprises exposing targeted tissue to a cumulative fluence greater than a threshold fluence at the time of treatment. In certain embodiments, the desired cumulative fluence for the treatment is greater than 35 J/cm². In certain embodiments, the desired cumulative fluence for the treatment is around 30-35 J/cm².

In certain embodiments, each pulse has a fluence between 0.1 J/cm² and 35 J/cm². In one embodiment, each pulse has a fluence around 22 J/cm². In one embodiment, each pulse has a fluence around 16 J/cm². In various embodiments, the fluence of each pulse can be predetermined based on the number of pulses that are used during a treatment period to reach a desired fluence.

In various embodiments, the beam of radiation can have a spot size between about 0.5 mm and about 25 mm, although larger and smaller spotsizes can be used depending on the application. In various embodiments the treatment area is larger than the spot size.

In various embodiments, the beam of radiation can have a pulse duration between about 10 μs and about 30 s, although larger and smaller pulse durations can be used depending on the application. In various embodiments, the beam of radiation can be delivered at a rate of between about 0.1 pulse per second and about 10 pulses per second, although faster and slower pulse rates can be used depending on the application. In one embodiment, the beam of radiation can have a pulse duration between about 0.1 second and about 20 seconds. In one embodiment, the beam of radiation can have a pulse duration between about 1 second and 20 seconds.

In various embodiments, the parameters of the radiation can be selected to deliver the beam of radiation to a predetermined depth. In some embodiments, the beam of radiation can be delivered to the target region about 0.5 mm to about 10 mm below an exposed surface of the skin, although shallower or deeper depths can be selected depending on the application. In one embodiment, the beam of radiation is delivered to the target region about 1 mm to about 10 mm below an exposed surface of the skin.

In various embodiments, the tissue can be heated to a temperature of between about 50° C. and about 80° C., although higher and lower temperatures can be used depending on the application. In one embodiment, the temperature is between about 55° C. and about 70° C.

To minimize unwanted thermal injury to tissue not targeted (e.g., an exposed surface of the target region and/or the epidermal layer), the delivery system 33 shown in FIG. 3 can include a cooling system for cooling before, during or after delivery of radiation, or a combination of the aforementioned. Cooling can include contact conduction cooling, evaporative spray cooling, convective air flow cooling, or a combination of the aforementioned. In one embodiment, the handpiece 36 includes a skin contacting portion that can be brought into contact with the skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

A spray cooling device can use cryogen, water, or air as a coolant. In one embodiment, a dynamic cooling device can be used to cool the skin (e.g., a DCD available from Candela Corporation). For example, the delivery system 33 shown in FIG. 3 can include tubing for delivering a cooling fluid to the handpiece 36. The tubing can be connected to a container of a low boiling point fluid, and the handpiece can include a valve for delivering a spurt of the fluid to the skin. Heat can be extracted from the skin by virtue of evaporative cooling of the low boiling point fluid. The fluid can be a non-toxic substance with high vapor pressure at normal body temperature, such as a Freon, tetrafluoroethane, or liquefied CO₂.

The time duration of cooling and of radiation application can be adjusted to maximize heating and thermal injury to the region proximate to the dermal interface. In tissue where the dermal interface is deeply situated, the cooling time can be lengthened such that cooling can be extended deeper into the skin. At the same time, the time duration of radiation application can be lengthened such that heat generated by the radiation in the region of dermis closer to the skin surface can be removed via thermal conduction and blood flow, thereby minimizing injury to the tissue overlying the dermal interface. Similarly if the dermis overlying the dermal interface is thin, the time duration of cooling and of radiation application can be adjusted to be shorter, such that thermal injury is confined to the region proximate to the dermal interface.

In various embodiments, a topical osmotic agent is applied to the region of skin to be treated, prior to treatment. The osmotic agent reduces the water content in the dermis overlying the dermal interface. This reduction in the water content can increase the transmission of the radiation into the dermal interface region and into the subcutaneous fat, thereby more effectively treating the area, reducing injury to the dermis, and reducing treatment pain. The osmotic agent can be glycerin or glycerol. A module can be used to apply the osmotic agent. The module can be a needle or syringe. The module can include a reservoir for retaining the osmotic agent and an injector for applying the agent to a skin region.

In various embodiments, a delivery system can include a focusing system for focusing the beam of radiation below the surface of the skin in the target region to affect at least one fat cell. The focusing system can direct the beam of radiation to the target region about 0.1 mm to about 10 mm below the exposed surface of the skin. In some embodiments, the delivery system can include a lens, a planoconvex lens, or a plurality of lens to focus the beam of radiation.

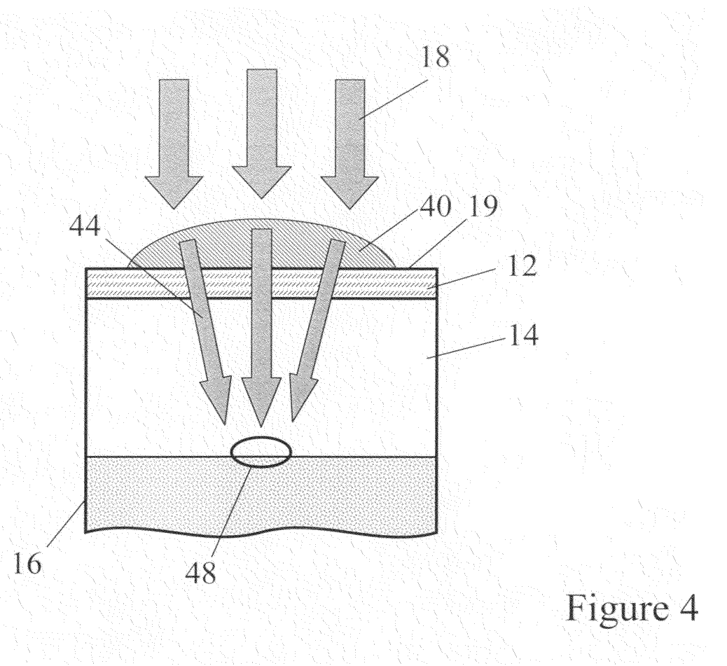

FIG. 4 shows a planoconvex lens 40 positioned on a surface 19 of a section of skin, including an epidermal region 12, a dermal region 14, and deep tissue 16. The planoconvex lens 40 focuses radiation 24 (focusing shown by arrows 44) to a sub surface focal region 48, which can include at least one fat cell. In certain embodiments, the element contacting the skin can be pressed into or against the skin to displace blood in the dermis, thereby increasing the transmission of the radiation through the dermis and reducing unwanted injury to the skin.

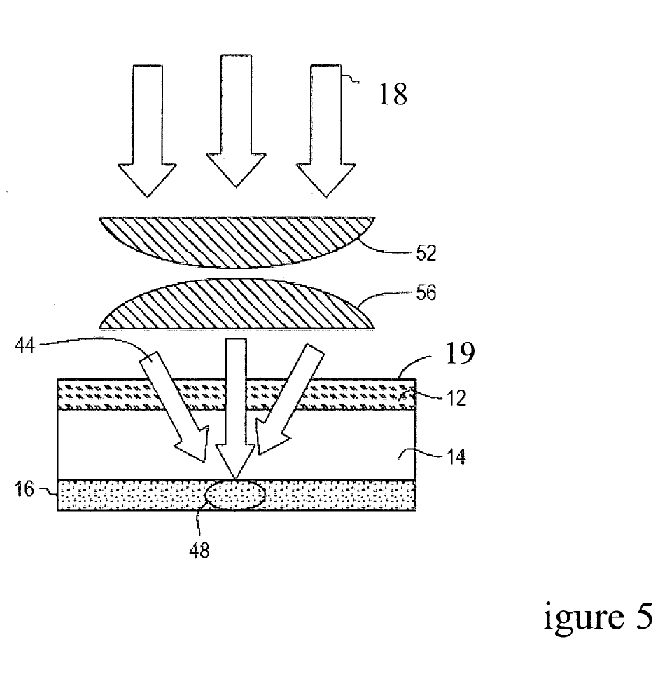

FIG. 5 shows a plurality of lens 52, 56 spaced from the skin surface 19. The plurality of lens 52, 56 focus the radiation 18 (focusing shown by the arrows 44) to the sub surface focal region 48.

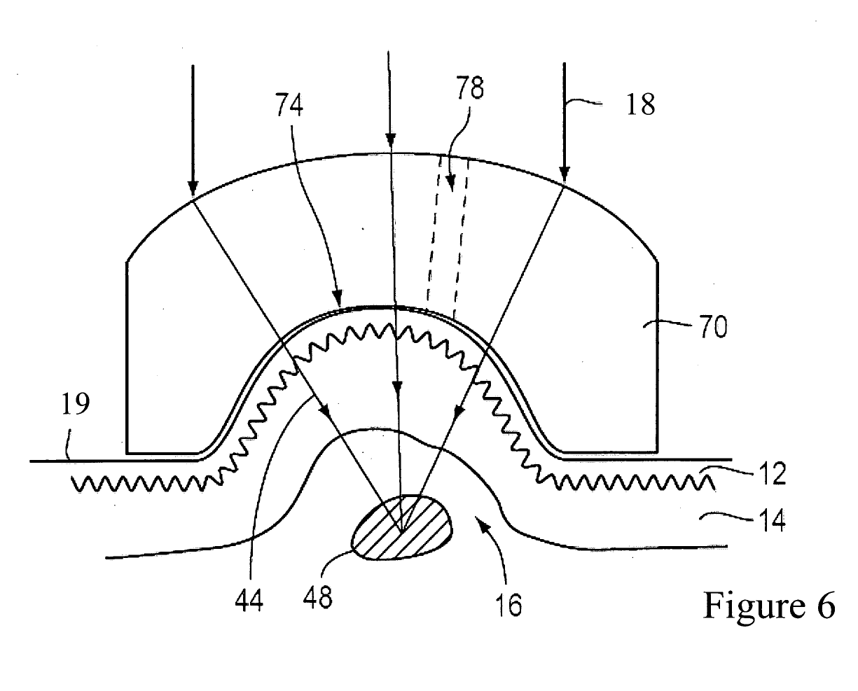

FIG. 6 shows a lens 70 having a concave surface 74 for contacting the skin surface 19. In certain embodiments, the lens 70 is placed proximate to a target region of skin. Vacuum can be applied to draw the target region of skin against the concave surface 74 of the lens 70. Vacuum can be applied through orifice 78 in the lens 70 by a vacuum device. The lens 70 focuses the radiation 18 to the sub surface focal region 48.

In various embodiments, the source of radiation can be a diode laser having sufficient power to affect one or more fat cells. An advantage of diode lasers is that they can be fabricated at specific wavelengths that target fatty tissue. A limitation, though, of many diode laser devices and solid state devices targeting fatty tissue is the inability to produce sufficient power to effectuate a successful treatment.

In one embodiment, a diode laser of the invention is a high powered semiconductor laser. In one embodiment, the source of radiation is a fiber coupled diode laser array. For example, an optical source of radiation can include a plurality of light sources (e.g., semiconductor laser diodes) each adapted to emit a beam of light from a surface thereof. A plurality of first optical fibers each can have one end thereof adjacent the light emitting surface of a separate one of the light sources so as to receive the beam of light emitted therefrom. The other ends of the first optical fibers can be bundled together in closely spaced relation so as to effectively emit a single beam of light, which is a combination of the beams from all of the first optical fibers. A second optical fiber can have an end adjacent the other ends of the first optical fibers to receive the beam of light emitted from the bundle of first optical fibers. The beam of light from the bundled other ends of the first optical fibers can be directed into the second optical fiber. The first optical fiber can have a numerical aperture less than that of the second fiber. An exemplary fiber coupled diode laser array is described in U.S. Pat. No. 5,394,492, owned by the assignee of the instant application and the entire disclosure of which is herein incorporated by reference.

In various embodiments, beams from multiple diode lasers or diode laser bars can be combined using one or more lens. In one embodiment, an array of diode lasers is mounted in a handpiece of the delivery system, and respective beams of radiation from each diode laser can be directed to the target region. The beams of radiation can be combined so that they are incident at substantially the same point. In one embodiment, the one or more lens direct the multiple beams of radiation into a single optical fiber. A handpiece of the delivery system projects the combined beam of radiation to the target region of skin.

In various embodiments, a laser diode array can include a plurality of discrete emitter sections mounted on a substrate, e.g., a laser bar. Each discrete emitter section can include a light emitting material having an active region and an inactive region. Each discrete emitter section can be a laser diode. The substrate provides electrical isolation between adjacent discrete emitter sections. A plurality of wire bonds can connect electrically the plurality of discrete emitter sections in a series configuration. Each discrete emitter section can be physically isolated from an adjacent discrete emitter section by, for example, mechanically dicing to remove a portion of the inactive region. In various embodiments, the light emitting material is a semiconductor material. Suitable semiconductor materials include InGaAlP, InGaP, InGaAs, InGaN, or InGaAsP. In one embodiment, the active region is InGaAs, and the inactive region is GaAs. In various embodiments, the substrate can be diamond, ceramic, BeO, alumina, or a gold plated ceramic. The light emitting material can be soldered to the substrate, e.g., using tin-containing solders such as SnBi, SnPb, and SnPbAg (e.g., Sn62) and gold-containing solders such as AuGe. An exemplary laser diode array is described in U.S. patent application Ser. No. 11/503,492 file Aug. 11, 2006, owned by the assignee of the instant application and the entire disclosure of which is herein incorporated by reference.

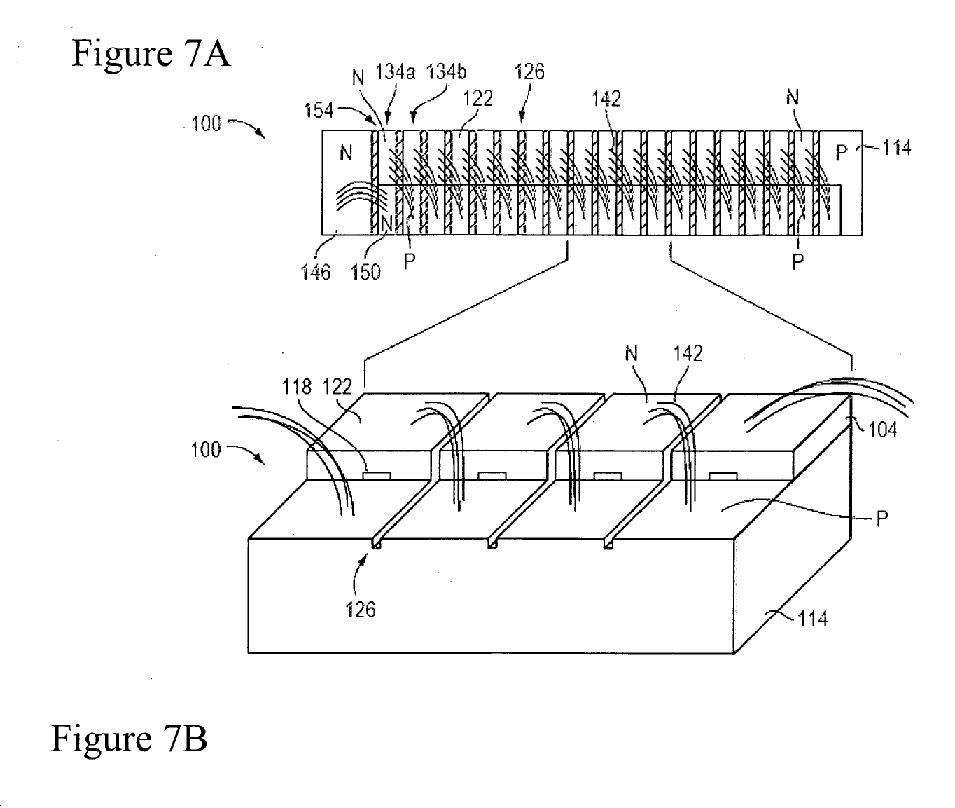

FIGS. 7A and 7B shows a laser diode array 100 including a light emitting material 104 formed on a substrate 114. The light emitting material 104 includes one or more active regions 118 and an inactive region 122. Cuts 126 can be positioned between adjacent active regions 118 to form a plurality of discrete emitter sections 134. Cuts 126 can be removal points or dicing points. Each discrete emitter section 134 can be electrically and/or physically isolated from an adjacent discrete emitter section. FIG. 7B shows a first n-type region 146 connected to a second n-type region 150 over an isolation cut 154 so that an operator can have a soldering point for connecting to a drive circuit. The remaining connections are formed between an n-type region and an adjacent p-type region. For example, a n-type region of a first discrete emitter section 134a of the light emitting material 104 can be electrically coupled to a p-type region of a second discrete emitter section 134b. The p-type region can be electrically coupled to a portion of the substrate 114, and the n-type region of the first discrete emitter section 134a can be connected to that substrate 114 portion. For example, FIG. 7B shows an enlarged view of four discrete emitter sections 134 of the laser diode array 100 where the wire 142 is bonded to the substrate 114.

In certain embodiments, a p-type region of a first discrete emitter section 134 of the light emitting material 104 can be electrically coupled to a n-type region of a second discrete emitter section 134. The n-type region can be electrically coupled to a portion of the substrate 114, and the p-type region of the first discrete emitter section 134 can be connected to that substrate 114 portion.

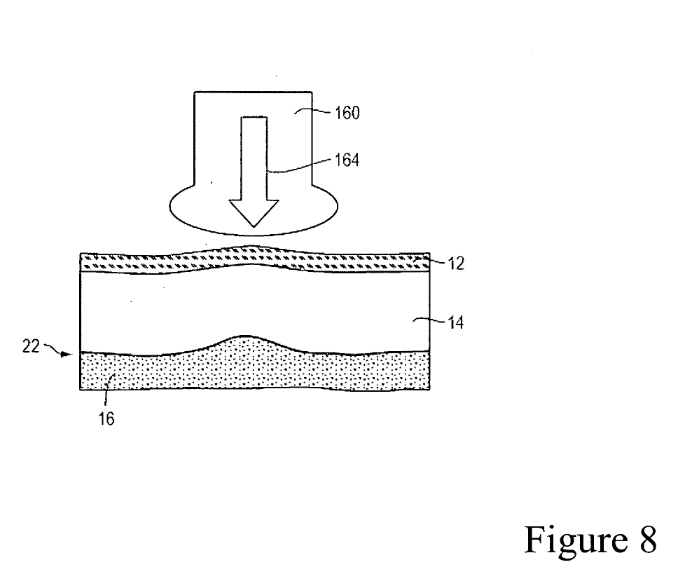

In various embodiments, an ultrasound device can be used to measure the depth or position of the fatty tissue. For example, a high frequency ultrasound device can be used. FIG. 8 shows a handpiece of an ultrasound device 160 placed proximate to the skin to make a measurement. In one embodiment, the ultrasound device 160 can be place in contact with the skin surface. The ultrasound device 160 can deliver ultrasonic energy 164 to measure position of the dermal interface 17, so that radiation can be directed to the interface 17 or to measure the position of targeted deep tissue 16, for instance, below the dermal interface.

The time duration of the cooling and of the radiation application can be adjusted so as to maximize the thermal injury to the vicinity of targeted tissue. For example, if the position of targeted fatty tissue is known, then parameters of the optical radiation, such as pulse duration and/or fluence, can be optimized for a particular treatment. Cooling parameters, such as cooling time and/or delay between a cooling and irradiation, can also be optimized for a particular treatment. Accordingly, a zone of thermal treatment can be predetermined and/or controlled based on parameters selected. For example, the zone of thermal injury can be positioned in or proximate to the dermal interface.

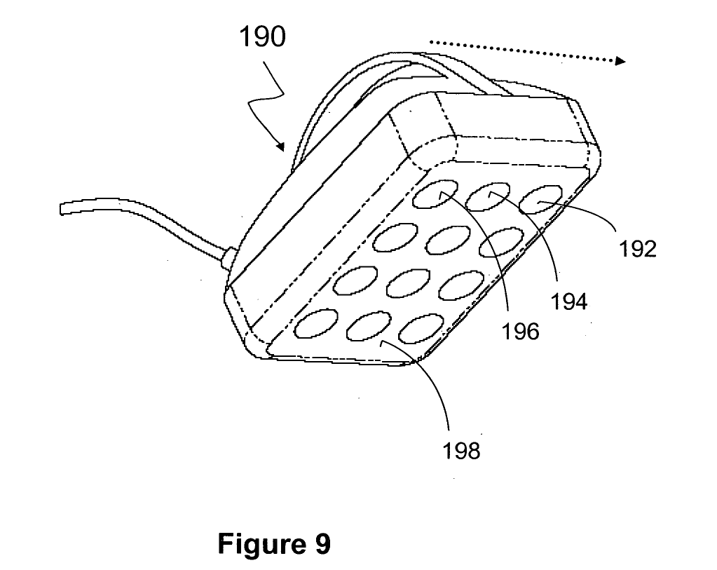

An alternative to moving a single beam in an exposure pattern, such as 26-28, is to provide a hand piece with multiple output beams rather than a single beam hand piece 36. FIG. 9 shows an exemplary hand piece having multiple beams. Hand piece 190 contains a radiation array 198 of output beams including beams 192, 194, and 196. The beams of hand piece 190 can result from separate laser diodes contained in hand piece 190, from incoherent light sources contained in hand piece 190, or from an energy source external to hand piece 190, such as energy source 32. The illustrative embodiment of hand piece 190 is a 3×4 array, but the layout of the radiation array 198 may also be any pattern, such as the shape of exposure patterns 26-28.

Hand piece 190 can be operated in accordance with certain embodiments by placing the hand piece 190 over an area of biological tissue to be treated. The beams of the radiation array 198 are then pulsed. Between subsequent pulses of the array 198, hand piece 190 is moved in the direction of the depicted arrow. Moving hand piece 190 between pulses of radiation array 198 exposes a region of tissue to beams 192, 194, and 196 sequentially. The sequential exposure or the tissue region to beams 192, 194, and 196 effectuates pulse stacking, resulting in therapeutic effects similar to the single beam exposure patterns, for example, shown in FIGS. 2A-2C.

Each exposed region of biological tissue, therefore, is exposed only to a portion of the total fluence delivered by the radiation source. The source pulse rate is faster than the effective exposure pulse rate that a region of biological tissue is exposed to the beam. In certain embodiments the duty cycle of the source pulse rate is greater than 50%, while the duty cycle of the effective exposure pulse rate for a given region of biological tissue is less than 50% because each region of biological tissue is exposed to a portion of the pulses. In certain embodiments the source pulse rate is less than or approximately 50%, while the duty cycle of the effective exposure pulse rate for a given region of biological tissue is substantially less than 50% because each region of biological tissue is exposed to a portion of the pulses. In certain embodiments the duty cycle of the source pulse rate varies based on operator control. In certain embodiments, each pulse is initiated by a manual or foot switch to turn on the radiation source. In certain embodiments each pulse is terminated automatically. In certain embodiments each pulse is terminated manually.

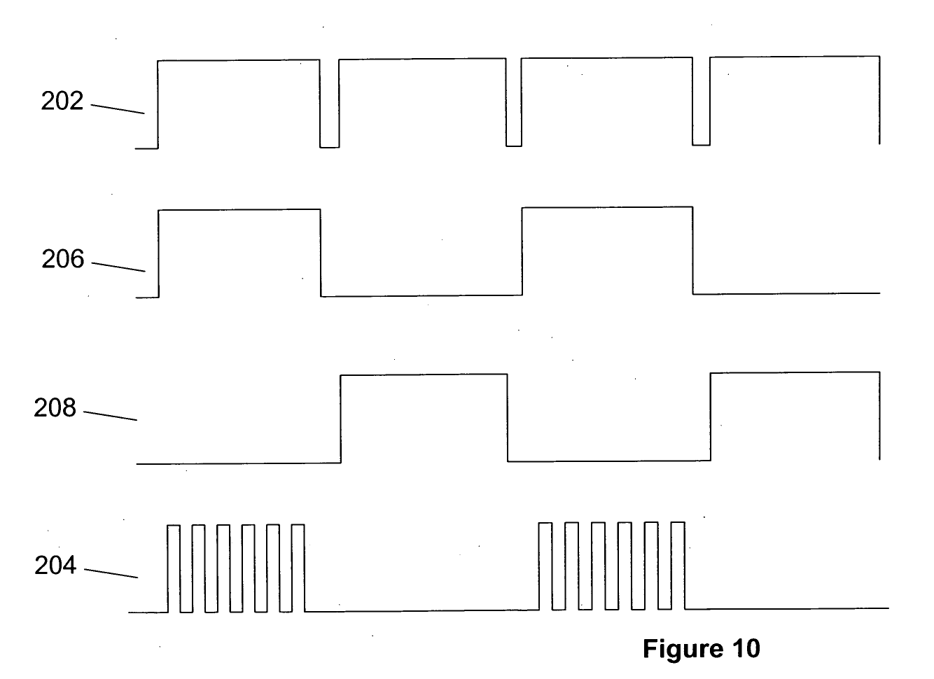

FIG. 10 illustrates the relationship between the pulse rate and duty cycle of the radiation beam and the exposure pulse rate and duty cycle for each region of tissue being exposed. The intensity 202 as a function of time of the radiation beam 18x can have a duty cycle greater than 50%, efficiently using the beam to expose tissue. This method can be used to expose at least one region of tissue most of the time during the treatment. The exposure intensity 206 as a function of time for a first tissue region 20a can have a duty cycle of less than 50%, allowing pulses to be stacked to create a larger cumulative fluence, while allowing the surface tissue to cool down for a period of time between exposure pulses. During a cooling period of the first tissue region 20a, a second tissue region 20b can be exposed to the radiation beam 18x as shown by intensity 208 as a function of time. In this example, there are only two regions, so every other pulse of radiation beam 18x exposes tissue region 20a and 20b, alternately. When another exposure pattern, such as exposure pattern 26-28, is used the number of pulses of radiation beam 18x before a region is exposed to another pulse corresponds to the pattern.

In certain embodiments, each pulse of radiation can be delivered in a series of short sub-pulses spaced in time such that within a region of biological tissue, the tissue is exposed to radiation intermittently over the pulse duration. Sub-pulses can range in duration from about 10 μs to the pulse duration. In certain embodiments, sub-pulses can be applied using a controlled duty cycle to control the fluence delivered during a pulse. In certain embodiments, sub-pulses can be applied using a controlled fluence to control the fluence delivered during a pulse, such as by way of pulse width modulation. Exposure intensity 204 as a function of time for tissue region 20a shows an example of how a tissue region can be exposed to a radiation beam comprising sub pulses.

In one illustrative embodiment, treatment of an area of tissue requires 40 seconds of radiation exposure using a 1 cm² beam spot. If the radiation beam were held over each region of biological tissue, the treatment speed is only 0.025 cm²/sec. However, using pulse stacking, the beam of radiation can be divided into 10 pulses of 1 second each spaced by 4 seconds between pulses, thereby delivering a exposure pulse rate of 0.25 pulse/sec with approximately a 25% duty cycle. As per this illustrative embodiment, one could treat a sequence of four 1 cm² spots sequentially with 1 second per pulse and then repeat the pattern 10 times. In this way, each lcm² spot is exposed to a sequence of 10 pulses separated by 4 seconds. The treatment speed using the illustrative embodiment is 4×1 cm² in 40 seconds, that is, 0.1 cm²/sec, four times the treatment speed of static pulse stacking.

Various embodiments may feature a kit suitable for use in the treatment of subcutaneous fat and/or cellulite, varicose veins, skin pigmentation, and skin rejuvenation. The kit can be used to improve the cosmetic appearance of a region of skin. The kit can include a source of a beam of radiation and instruction means. The instruction means can include instructions for directing the beam of radiation to a deep tissue zone. The beam of radiation thermally affect at least one cell in the deep tissue zone without causing substantial unwanted injury to the epidermal region and cause thermal injury to a dermal region to induce collagen formation to strengthen the target region of skin. The source can include a fiber coupled laser diode array. A cooling system can be used to cool an epidermal region of the target region to minimize substantial unwanted injury thereto. The instruction means can prescribe a wavelength, fluence, pulse duration and/or and pattern form moving the beam between successive pulse for treatment of the subcutaneous fat region. The instruction means, e.g., treatment guidelines, can be provided in paper form, for example, as a leaflet, booklet, book, manual, or other like, or in electronic form, e.g., as a file recorded on a computer readable medium such as a drive, CD-ROM, DVD, or the like.

In some embodiments, the instruction means can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site.

The instruction means can be performed by one or more programmable processors executing a computer program to perform functions of the invention by operating on input data and generating output. The instruction means can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks.

The computing system can include clients and servers. A client and a server are generally remote from each other and typically interact through a communication network. The relationship of client and server arises by virtue of computer programs running on the respective computers and having a client-server relationship to each other.

While the invention has been particularly shown and described with reference to specific illustrative embodiments, it should be understood that various changes in form and detail may be made without departing from the spirit and scope of the invention.

What is claimed:

1. A method of applying energy to a plurality of regions of a biological tissue, comprising:
exposing a first region of the biological tissue to an electromagnetic beam for a first selected length of time;
positioning the electromagnetic beam over a second region of the biological tissue;
exposing the second region of the biological tissue to the electromagnetic beam for a second selected length of time;
subsequently positioning the electromagnetic beam over the first region of the biological tissue; and
exposing the first region of the biological tissue to the electromagnetic beam for a third selected length of time.

2. The method of claim 1 wherein the steps of positioning and exposing are repeated for a fourth selected length of time.

3. The method of claim 1 wherein the steps of positioning and exposing are repeated such that the first region of the biological tissue is exposed a selected number of times.

4. The method of claim 1 further comprising cooling the first region of the biological tissue and the second region of the biological tissue between successive exposures to the electromagnetic beam.

5. The method of claim 1 wherein the steps of positioning and exposing are repeated such that multiple adjacent regions of biological tissue are exposed in a predetermined sequence.

6. The method of claim 1 wherein the steps of exposing damage at least one fat cell.

7. The method of claim 1 wherein the steps of exposing cause partial denaturation of collagen fibers in a dermal zone of the first region of biological tissue.

8. A method of applying energy to a plurality of regions of a biological tissue, comprising:
exposing a first region of the biological tissue to an electromagnetic beam for a first length of time, the first region including a first tissue zone overlying a second tissue zone;
positioning the electromagnetic beam over a second region of the biological tissue;
exposing the second region of the biological tissue to the electromagnetic beam for a second length of time, during which the first tissue zone cools substantially more than the second tissue zone;
repositioning the electromagnetic beam over the first region of the biological tissue; and
re-exposing the first region of the biological tissue to the electromagnetic beam before the second tissue zone cools below a threshold temperature.

9. The method of claim 8 wherein the second region of the biological tissue includes a third tissue zone overlying a fourth tissue zone.

10. The method of claim 9 further comprising:
repositioning the electromagnetic beam over the second region of the biological tissue; and
re-exposing the second region of the biological tissue to the electromagnetic beam before the fourth tissue zone cools below the threshold temperature.

11. The method of claim 8 wherein the steps are repeated until a temperature of the second biological tissue zone exceeds a threshold value.

12. The method of claim 8 wherein the steps are repeated for a predetermined number of times.

13. The method of claim 8 wherein the first length of time is substantially the same length as the second length of time.

14. The method of claim 8 further comprising cooling a surface of the biological tissue.

15. The method of claim 8 wherein the steps of positioning and repositioning are repeated such that multiple adjacent regions of biological tissue are exposed in a predetermined sequence.

16. The method of claim 8 wherein the steps of exposing and re-exposing damage at least one fat cell.

17. The method of claim 8 wherein the steps of exposing and re-exposing cause partial denaturation of collagen fibers in the first tissue zone.

18. A method for applying energy to a first biological tissue that has a longer thermal relaxation time than a second biological tissue which is above the first biological tissue, comprising:
exposing a first region of the first and the second biological tissues to an electromagnetic beam for a first length of time, wherein the first length of time is short enough to prevent a temperature of the first region of the second biological tissue from substantially exceeding a first threshold value;
exposing a second region of the biological tissues to the electromagnetic beam for the first length of time, while the temperature of the first region of the second biological tissue decreases;
exposing the first region of the biological tissues to the electromagnetic beam for the first length of time, while a temperature of the second region of the second biological tissue decreases;
repeatedly performing the exposing steps such that a temperature of the first region of the first biological tissue and a temperature of the second region of the first biological tissue exceeds a second threshold value, whereas the temperature of the first region of the second biological tissue and the temperature of the second region of the second biological tissue do not substantially exceed the first threshold value.

19. The method of claim 18 further comprising cooling the first and second regions of biological tissue between successive exposures to the electromagnetic beam.

20. The method of claim 18 wherein the steps of exposing damage at least one fat cell.

21. The method of claim 18 wherein the steps of exposing cause partial denaturation of collagen fibers in the first region of the second biological tissue.

22. The method of claim 18 wherein the steps of exposing are repeated such that multiple adjacent regions of biological tissue are exposed in a predetermined sequence.
